(19) United States
(12) Patent Application Publication
(10) Pub. No.: US 2008/0234324 A1
(43) Pub. Date: Sep. 25, 2008

(54) Piperidine Derivatives as Gcs Inhibitors

(76) Inventors: Michael Glen Orchard, Oxfordshire (GB); David Ian Carter Scopes, Oxfordshire (GB)

Correspondence Address: DICKSTEIN SHAPIRO LLP, 1177 AVENUE OF THE AMERICAS (6TH AVENUE), NEW YORK, NY, 10036-2714, US

(21) Appl. No.: 10/586,188

(22) PCT Filed: Jan. 11, 2005
(86) PCT No.: PCT/GB2005/000071

§ 371 Date: Jul. 14, 2006

(30) Foreign Application Priority Data
Jan. 14, 2004 (GB) 0400812.4

(51) Int. Cl. A61K 31/451; C07D 211/44; A61P 35/00; A61P 3/04; A61P 29/00

(52) U.S. Cl. 514/328; 546/219

(57) ABSTRACT

The present invention relates to novel piperidine derivatives that are useful as inhibitors of glucosylceramide synthase (GCS) for use in the treatment and prevention of diseases or disorders mediated by GCS, including cancer, infectious diseases, neuronal disorders or injury and inflammatory diseases. The invention further relates to the pharmaceutical composition comprising the novel piperidine derivatives and methods for preparing the same.

The present invention relates to novel piperidine derivatives useful as inhibitors of glucosylceramide synthase (GCS; UDP-glucose:ceramide glucosyltransferase, UDP-glucose:N-acylsphingosine D-glucosyltransferase, EC 2.4.1.80), methods for their preparation and their use in medicine, specifically in the treatment and prevention of disease states mediated by GCS. The compounds find use in the treatment of glycolipid storage diseases, diseases associated with glycolipid accumulation, cancers in which glycolipid synthesis is abnormal, infectious diseases caused by organisms which use cell surface glycolipids as receptors, infectious diseases in which synthesis of glucosylceramide is essential or important, diseases in which excessive glycolipid synthesis occurs, neuronal disorders, neuronal injury and inflammatory diseases or disorders associated with macrophage recruitment and activation.

GCS is an intracellular enzyme that catalyzes the assembly of uridine diphosphate-glucose and ceramide into the glycolipid, glucosylceramide. The role of GCS in regulating ceramide levels has been explored, since this molecule can induce apoptotic cell death (J. Biol. Chem., 2000, 275(10), 7138-43). The role of GCS in maintaining cholesterollglycolipid ‘rafts’, cell-surface membrane domains of specialized permeability and functionality that appear to be involved in a variety of signal transduction events, has also been investigated (Nature, 1997, 387(6633), 569-72).

GCS is considered to be a target for treating certain human diseases. Glucosylceramide and structurally related glycolipids are stored in the lysosomes of patients with genetic diseases, which result from a mutation in one of the essential glycolipid-degrading enzymes (e.g. Gaucher, Tay Sachs, Sandhoffs, GM1 gangliosidosis and Fabry diseases). Glycolipid storage also occurs as a secondary effect in some tissues (e.g. neuronal tissue) with genetic storage diseases such as Niemann-Pick C disease, mucopolysaccharidoses, mucolipidosis type IV (Proc. Natl. Acad. Sci. USA, 1998, May 26, 95(11), 6373-8) and α-mannosidosis (Proc. Natl. Acad. Sci. USA, 1991, Dec. 15, 88(24), 11330-4). GCS inhibitors may be applied to reduce the rate of glycolipid synthesis in diseased cells so that there is less glycolipid present to be stored, a treatment approach termed substrate deprivation. Studies have demonstrated that GCS inhibitors can be used to reduce the glycolipid accumulation seen in cell and animal models of glycolipid storage disorders (Proc. Natl. Acad. Sci. USA, 1999, 96(11), 6388-93; Science, 1997, 276(5311), 428-31; J. Clin. Invest., 2000, 105(11), 1563-71). Furthermore, clinical trials have shown that GCS inhibitors, such as, N-butyldeoxynojirimycin (NB-DNJ) are useful in treating human patients with Gaucher disease (Lancet, 2000, 355(9214), 1481-5). The use of the imino sugar NB-DNJ as a GCS inhibitor is disclosed in EP-A-0698012. EP-A-0536402 and EP-A-0698012 disclose that N-alkyl derivatives of deoxygalactonojirimycin, e.g. N-butyldeoxygalactonojirimycin (NB-DGJ), may also be of use in the treatment of glycolipid storage disorders.

The use of GCS inhibitors in the treatment of human malignancies has also been proposed. Tumours can synthesize abnormal quantities of glycolipids that are typically present/absent in normal tissues. In addition glycolipids, or gangliosides, in particular are shed by tumour cells and released into the extracellular space and the bloodstream. Both tumour shed and cell surface bound tumour gangliosides can influence tumour host cell interactions such as cell-cell contacts or adhesion (Methods Enzymol., 2000, 312, 447-58), cell motility (Mol. Chem. Neuropathol., 1995, 24(2-3), 121-35), growth factor signalling events (J. Biol. Chem., 2000, 275(44), 34213-23), tumour stimulated angiogenesis (Acta. Oncol., 1997, 36(4), 383-7) and tumour specific immune responses (J. Immunol., 1999, Oct. 1, 163(7), 3718-26). All these events can affect tumour development and progression. Glycolipids, glucosylceramide in particular, are known to accumulate in multidrug resistant (MDR) tumour cells (Anticancer Res., 1998, 18(1B), 475-80) and in vitro treatment of these cells with GCS inhibitors can reverse the MDR phenotype (J. Biol. Chem., 1997, 272(3), 1682-7; Br. J. Cancer, 1999, 81(3), 423-30).

WO99/24401 discloses N-substituted-deoxynojirimycin and -deoxygalactonojirimycin for use in the prevention of multidrug resistance in patients undergoing treatment with a chemotherapeutic agent. The only examples of N-arylalkyl substituents which are provided are phenylmethyl, 3-phenylpropyl, 6-phenylhexyl and 3-(4-methyl)phenylpropyl. WO99/24401 expresses a preference for the N-alkyl substituents n-butyl and n-hexyl.

WO92/00277 discloses the following compounds as carcinostatic agents:
3,4,5-piperidinetriol, 1-[(1,1′-biphenyl)-4-ylmethyl]-2-(hydroxymethyl)-, (2R,3S,4R,5S);
3,4,5-piperidinetriol, 2-(hydroxymethyl)-1-[(4-methoxyphenyl)methyl]-, (2R,3S,4R,5S);
3,4,5-piperidinetriol, 2-(hydroxymethyl)-1-[(4-methylthiophenyl)methyl]-, (2R,3S,4R,5S);
acetamide, N-[4-[[3,4,5-trihydroxy-2-ydroxymethyl)-1-piperidinyl]methyl]phenyl]-, (2R,3S,4R,5S); and
3,4,5-piperidinetriol, 2-(hydroxymethyl)-1-[(4-methoxy-3-methylphenyl)methyl]-, (2R,3S,4R,5S).

Cell surface glycolipids also have roles in infectious disease, serving as receptors for the binding of pathogenic bacteria (APMIS, 1990, December, 98(12), 1053-60, Review), fungi (Infect. Immun., 1990 July, 58(7), 2085-90) and viruses (FEBS Lett., 1984, May 7, 170(1), 15-18). In addition, glycolipids on the surface of cells are bound by bacterial toxins (Methods Enzymol., 2000, 312, 459-73) for instance, the B subunit of cholera toxin (ganglioside GM1) and verocytotoxin (globotriaosylceramide GB3) (J. Infect. Dis., 2001, suppl. 70-73, 183).

GCS inhibitors may also find use in the treatment of viral infections.

The use of GCS inhibitors may also be appropriate in a number of other clinical indications which are associated with abnormalities in glycolipid synthesis. Atherosclerotic lesions of human aorta have a higher ganglioside content than unaffected regions of the aorta and serum ganglioside concentrations in atherosclerotic patients are higher than in normal individuals (Lipids, 1994, 29(1), 1-5). Tissue derived from the kidneys of patients with polycystic kidney disease contains high levels of both glucosylceramide and lactosylceramide (J. Lipid. Res., 1996, June, 37(6), 1334-44). Renal hypertrophy in an animal model of diabetes is associated with increases in glycolipid synthesis, (J. Clin. Invest., 1993, March, 91(3), 797-803).

Glycolipid metabolism also plays a critical role in neuronal disorders, such as Alzheimer's disease and epilepsy. For instance, Niemann-Pick C(NPC) patient neurons present with fibrillar tangles reminiscent of the morphology seen in Alzheimer's disease.

GM1 ganglioside binding by amyloid beta-protein induces conformational changes that support its formation of fibrous polymers and the fibrillar deposition of this protein is an early event in Alzheimer's disease (Yanagisawa et al., 1995, Nat. Med. 1, 1062-6; Choo-Smith et al., 1997, Biol. Chem., 272, 22987-90). Thus, decreasing GM1 synthesis by using agents such as GCS inhibitors, e.g. NB-DNJ, could inhibit the fibre formation seen in Alzheimer's disease.

In contrast, preliminary clinical trials have shown that neurodegenerative processes seen in Parkinson's disease, stroke and spinal cord injuries seem to improve by treating patients with GM1 ganglioside (Alter, (1998), Ann. NY Acad. Sci., 845, 391-4011; Schneider, 1998, Ann. NY. Acad. Sci., 845, 363-73; Geisler, (1998), Ann. NY. Acad. Sci., 845, 374-81). It is possible that co-administering glucosylceramide synthesis inhibitors would provide the clinician greater control over this treatment course. GCS inhibitors like NB-DNJ would limit patient-specific inconsistencies by blocking their neuronal glycolipid synthesis. In addition, inhibiting glucosylceramide synthesis would limit the metabolism of administered glycolipids into other, perhaps unproductive, forms. Thus, the ability to modulate glucosylceramide synthesis with GCS inhibitors may be useful in the treatment of a wide variety of neuronal disorders.

In addition, it has also been shown that imino sugars can reversibly induce male sterility (van der Spoel, A. C. et al., Proc. Natl. Acad. Sci. USA, 2002, 99(26), 17173-8) and can, therefore, be used as male contraceptives. Also, GCS inhibitors could be used for the treatment of obesity.

A role for glycolipids in some aspects of inflammatory or immune responses has also been suggested. Following an inflammatory stimulus, such as that obtained with thioglycolate, the ganglioside profile of murine peritoneal macrophages changes from a simple profile (3 major species) in resting macrophage to a more complex profile (more than 14 species) in activated and recruited macrophage, see Ryan, J. L. et al., Yale J. Biol. Med., 1985, 58(2) 125-31; Yohe, H. C. et al., Biochim. Biophys. Acta., 1985, 818(1), 81-6; Yohe, H. C. et al., Immunol., 1991, 146(6), 1900-8. Furthermore, in vivo administration of an inflammatory agent, e.g. bacterial endotoxin, results in the increased expression of two enzymes, serine palmitoyltransferase and glucosylceramide synthase, which are key to the de novo synthesis of glycolipids, see Memon, R. A. et al., J. Biol. Chem., 1999, 274(28), 19707-13; Memon, R. A. et al., J. Lipid. Res., 2001, 42(3), 452-9.

Such a role for glycolipids is further supported by the demonstration of changes in glycolipid expression in animals with genetic defects which result in hyper- or hypo-sensitive responses to inflammatory stimuli. For example, upon endotoxin treatment in C3H/HeJ mice, which have a toll-like receptor 4 mutation and are hypo-responsive to bacterial endotoxin, recruited macrophages were found to lack ganglioside GM1b, which is a major ganglioside found in recruited macrophages in normal mice, see Yohe, H. C. et al., Immunol., 1991, 146(6), 1900-8; Yohe, H. C. et al., Immunol., 1986, 137(12), 3921-7.

Hence, GCS inhibitors may be useful in the treatment of inflammatory diseases and other disorders associated with macrophage recruitment and activation, including but not limited to, rheumatoid arthritis, Crohn's disease, asthma and sepsis.

WO02/055498 discloses piperidine derivatives useful as GCS inhibitors.

PCT/GB2003/003244 and PCT/GB2003/003099 (published after the priority date of the present application) disclose N-substituted -[(2S,3S,4R,5S)-3,4,5-trihydroxy-2-(hydroxymethyl)-1-piperidinyl] and -[(2S,3R,4R,5S)-3,4,5-trihydroxy-2-(hydroxymethyl)-1-piperidinyl] derivatives respectively as GCS inhibitors.

Given the importance of GCS in a wide spectrum of diseases, it is essential that new tools that provide a means for modulating this enzme's function be developed. Towards this end, we have identified a class of novel compounds that are useful in inhibiting GCS's catalytic activity.

The compounds of the invention may exhibit improved potency and/or selectivity for GCS, relative to non-lysosomal-β-glucocerebrosidase activity, over known hydroxylated piperidine derivatives.

According to the invention there is provided a compound of formula (I) or a pharmaceutically acceptable salt or prodrug thereof:

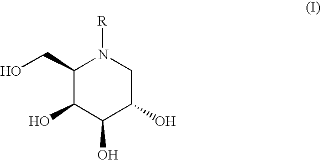

wherein

R is C₁₋₃ alkylAr¹ where Ar¹ is phenyl or pyridyl;

wherein phenyl is substituted by one or more substituents selected from CN, CON(R¹)₂, SOR², SO₂N(R¹)₂, N(R)₂, N(R¹)COR², N(R¹)SOR², C₀₋₆ alkylAr², C₂₋₆ alkenylAr² and C₃₋₆ alkynylAr² wherein one or more of the —CH₂— groups of the alkyl chain may be replaced with a heteroatom selected from O, S and NR³, provided that when the heteroatom is 0, at least two —CH₂— groups separate it from any additional O atom in the alkyl chain; or two adjacent substituents on the Ar¹ phenyl may together form a fused 5- or 6-membered saturated or unsaturated ring wherein the ring optionally contains 1 or 2 heteroatoms selected from O, S and NR³ and is optionally substituted by one or more substituents selected from, an oxo group, C₁₋₆ alkyl and C₀₋₃ alkylAr⁴;

and the Ar¹ phenyl is optionally substituted by one or more additional substituents selected from F, Cl, Br, CF₃, OCF₃, OR³ and C₁₋₆ alkyl;

and wherein pyridyl is substituted by one or more substituents, selected from, CN, CON(R¹)₂, SOₙR², SO₂N(R¹)₂, N(R⁵)₂, N(R¹)COR², N(R¹)SOₙR², F, Cl, Br, CF₃, OCF₃, OR³, C₁₋₄ alkyl, C₀₋₆alkylAr², C₂₋₆ alkenylAr² and C₃₋₆ alkynylAr² wherein one of the —CH₂— groups of the alkyl chain may be replaced with a heteroatom selected from O, S and NR³, provided that when the heteroatom is 0, at least two —CH₂— groups separate it from any additional O atom in the alkyl chain; or two adjacent substituents on the Ar¹ pyridyl may together form a fused 5- or 6-membered saturated or unsaturated ring wherein the ring optionally contains 1 or 2 heteroatoms selected from O, S and NR⁴ and is optionally substituted by one or more substituents selected from, an oxo group, C₁₋₆ alkyl and C₀₋₃ alkylAr⁴;

R¹ is H, C₁₋₄ alkyl optionally substituted by OH, Ar³, or C₁₋₆ alkylAr³, or the group N(R¹)₂ may form a 5- to 10-membered heterocyclic group optionally containing one or more additional heteroatoms selected from O, S and NR³ and is optionally substituted by an oxo group;

R² is C₁₋₆alkyl optionally substituted by OH, Ar³, or C₁₋₆alkylAr³;

R³ is H, or C₁₋₆ alkyl;

R⁴ is H, C₁₋₄ alkyl or C₀₋₃ alkylAr⁴;

R⁵ is H, C₁₋₆ alkyl optionally substituted by OH, Ar³ or C₁₋₆ alkylAr³, or the group N(R⁵)₂ may form a 5- to 10-membered heterocylic group optionally containing one or more additional heteroatoms selected from O, S and NR³ and is optionally substituted by an oxo group;

Ar² and Ar³ are independently phenyl or a 5- to 10-membered heteroaryl group containing up to 3 heteroatoms selected from O, S and NR³, which may be optionally substituted by one or more substituents selected from F, Cl, Br, CN, CF₃, OCF₃, OR³ and C₁₋₆ alkyl;

Ar⁴ is phenyl or pyridyl either of which may be optionally substituted by one or more substituents selected from F, Cl, Br, CN, CF₃, OCF₃, OR³ and C₁₋₆ alkyl; and

n is 0, 1 or 2;

provided that the compound is not:

a) 3,4,5-piperidinetriol, 1-[(1,1′-biphenyl)-4-ylmethyl]-2-(hydroxymethyl)-, (2R,3S,4R,5S);

b) 3,4,5-piperidinetriol, 2-(hydroxymethyl)-1-[(4-methoxyphenyl)methyl]-, (2R,3S,4R,5S);

c) 3,4,5-piperidinetriol, 2-(hydroxymethyl)-1-[(4-methylthiophenyl)methyl]-, (2R,3S,4R,5S);

d) acetamide, N-[4-[[3,4,5-trihydroxy-2-(hydroxymethyl)-1-piperidinyl]methyl]phenyl]-, (2R,3S,4R,5S); or

e) 3,4,5-piperidinetriol, 2-(hydroxymethyl)-1-[(4-methoxy-3-methylphenyl)methyl]-, (2R,3S,4R,5S).

R is preferably C₁ alkylAr¹.

Ar¹ is preferably phenyl, wherein phenyl is substituted as defined for formula (I).

Ar¹ phenyl is preferably substituted on the para position.

More preferably Ar¹ is phenyl, wherein phenyl is substituted by one or more substituents selected from CN, CON(R¹)₂, SO₂N(R¹)₂, N(R⁵)₂, N(R¹)COR², C₀₋₆ alkylAr² and C₂₋₆alkenylAr² wherein one or more of the —CH₂— groups of the alkyl chain may be replaced with a heteroatom selected from O, S and NR³, provided that when the heteroatom is O, at least two —CH₂— groups separate it from any additional O atom in the alkyl chain; or two adjacent substituents on the Ar¹ phenyl may together form a fused 5- or 6-membered saturated or unsaturated ring wherein the ring optionally contains 1 or 2 heteroatoms selected from O and NR⁴ and is optionally substituted by one or more substituents selected from, an oxo group, C₁₋₆ alkyl and C₀₋₃ alkylAr⁴, and the Ar¹ is optionally substituted by one or more additional substituents selected from F, Cl, Br, CF₃, OCF₃, OR³ and C₁₋₆ alkyl.

Yet more preferably Ar¹ is phenyl, wherein phenyl is substituted by one or more substituents selected from CN, CON(R¹)₂, N(R⁵)₂ and C₀₋₆alkylAr² herein one or more of the —CH₂— groups of the alkyl chain may be replaced with a heteroatom selected from O, S and NR³, provided that when the heteroatom is O, at least two —CH₂— groups separate it from any additional O atom in the alkyl chain; or two adjacent substituents on the Ar¹ phenyl may together form a fused 5- or 6-membered saturated or unsaturated ring wherein the ring optionally contains 1 or 2 heteroatoms selected from O and NR⁴ and is optionally substituted by one or more substituents selected from, an oxo group, C₁₋₆ alkyl and C₀₋₃ alkylAr⁴, and the Ar¹ is optionally substituted by one or more additional substituents selected from F, Cl, Br, CF₃, OCF₃, OR³ and C₁₋₆alkyl.

Even more preferably Ar¹ is phenyl, wherein phenyl is substituted by one or more substituents selected from CN, CON(R¹)₂, N(R⁵)₂ and C₀₋₆alkylAr² wherein one or more of the —CH₂— groups of the alkyl chain may be replaced with O, provided that at least two —CH₂— groups separate it from any additional O atom introduced into the alkyl chain, and the Ar¹ phenyl is optionally substituted by one or more additional substituents selected from F, Cl, Br, CF₃, OCF₃, OR³ and C₁₋₆alkyl.

When Ar¹ is phenyl and has an additional optional substitutent as defined for formula (I) on the ortho position, the substituent is preferably selected from OCH₃ and F. More preferably the ortho substituent is F.

When Ar¹ is phenyl substituted by C2 alkylAr² wherein one of the —CH₂— groups of the alkyl chain is replaced with O, preferably, the —CH₂— group linked to the Ar¹ phenyl is replaced with O.

R¹ is preferably H, C₁₋₆alkyl or C₁₋₆alkylAr³. More preferably R¹ is H or C₁₋₆alkylAr³.

R² is preferably Ar³ or C₁₋₆alkylAr³. More preferably R² is C₁₋₆alkylAr³.

R³ is preferably H.

R⁴ is preferably H or C₁₋₆alkyl. More preferably R⁴ is H.

R⁵ is preferably C₁₋₆alkyl optionally substituted by OH, or C₁₋₆alkylAr³. More preferably R⁵ is C₁₋₆ alkyl.

For the groups R¹, R² or R⁵, the group C₁₋₆alkylAr³ is preferably C₁₋₃alkylAr³, for example, C₁ alkylAr³ or C₂ alkylAr³.

Ar² is preferably phenyl which may be optionally substituted by one or more substituents selected from F, Cl, Br, CN, CF₃, OCF₃, OR³ and C₁₋₆ alkyl.

Ar³ is preferably phenyl which may be optionally substituted by one or more substituents selected from F, Cl, Br, CN, CF₃, OCF₃, OR³ and C₁₋₆ alkyl.

Ar⁴ is preferably phenyl which may be optionally substituted by one or more substituents selected from F, Cl, Br, CN, CF₃, OCF₃, OR³ and C₁₋₆ alkyl.

n is preferably 2.

In the groups CON(R¹)₂, SO₂N(R¹)₂, and N(R⁵)₂ the R¹ and R⁵ groups may be the same or different.

When two adjacent substituents on the Ar¹ form a fused 5- or 6-membered saturated or unsaturated ring optionally containing 1 or 2 heteroatoms selected from O, S and NR⁴, examples of bicyclic groups which may be formed include benzofuran, indole, benzoxazine, quinoline and isoquinoline.

When N(R¹)₂ forms a 5- to 10-membered heterocyclic group optionally containing one or more additional heteroatoms selected from O, S and NR³, examples of heterocyclic groups include, piperidine, piperazine, morpholine and quinoline.

When NR⁵)₂ forms a 5- to 10-membered heterocyclic group, preferably a 5- or 6-membered heterocyclic group, optionally containing one or more additional heteroatoms selected from O, S and NR³, examples of heterocyclic groups include, piperidine, piperazine and morpholine.

When Ar² or Ar³ is a 5- to 10-membered heteroaryl group, examples of heteroaryl groups include furan, thiophene, oxazole, triazole, pyridine, pyrazine, pyrimidine, benzofuran benzothiophene and benzoxazine.

The compounds of the invention preferably have a molecular weight of less than 800, more preferably less than 600.

The term “alkyl” as used herein whether on its own or as part of a larger group e.g. “alkylaryl”, includes both straight and branched chain radicals. The term alkyl also includes those radicals wherein one or more hydrogen atoms are replaced by fluorine. Alkenyl and alkynyl are to be interpreted accordingly.

The term “heterocyclic group” as used herein includes, unless otherwise defined, non-aromatic and aromatic, single and fused, rings containing one or more, e.g. up to three, heteroatoms in each ring, each of which is selected from O, S and N, which rings, may be unsubstituted or substituted. Each heterocyclic ring suitably has from 5 to 10, preferably 5, 6, 9 or 10 ring atoms. A fused heterocyclic ring system may include carbocyclic rings and need include only one heterocyclic ring. Examples of heterocyclyl groups, including heteroaromatic ring systems, are as follows: pyrrolidine, piperidine, piperazine, morpholine, imidazolidine, pyrazolidine, pyrrole, quinoline, isoquinoline, pyridine, pyrazine, pyrimidine, oxazole, thiazole, thiophene, indole, fulran, thiadiazole, triazole, imidazole, benzopyran, benzofuran, benzothiophene, benzoxazine and benzamidazole. “Heteroaryl” is to be interpreted accordingly.

Specific compounds of the invention include the compounds provided in the Examples and pharmaceutically acceptable salts and prodrugs thereof.

Preferred compounds of the invention include those provided in the Examples and pharmaceutically acceptable salts and prodrugs thereof.

As described herein, for all aspects of the invention, reference to compounds of formula (I) encompasses pharmaceutically acceptable salts and prodrugs thereof.

As described herein, the compounds of the present invention can be used for the inhibition of GCS. Thus, an aspect of the present invention provides the use of the compounds of formula (I) in medicine.

Suitable, pharmaceutically acceptable salts of the compounds of formula (I) include, but are not limited to, salts with inorganic acids such as hydrochloride, sulfate, phosphate, diphosphate, hydrobromide and nitrate, or salts with an organic acid such as malate, maleate, fumarate, tartrate, succinate, citrate, acetate, lactate, methanesulfonate, p-toluenesulfonate, palmitate, salicylate and stearate.

Suitable prodrugs of the compounds of formula (I) include, but are not limited to, pharmaceutically acceptable esters such as C₄ allyl esters.

Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain of the R groups of compounds of formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

The compounds of the invention may exist as tautomers, e.g. keto/enol tautomers, all of which are included within the scope of formula (I).

Since the compounds of formula (I) are intended for use in pharmaceutical compositions it will readily be understood that they are preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5%, e.g. 10 to 59%, of a compound of formula (I) or pharmaceutically acceptable derivative thereof.

The compounds of formula (I) can be prepared by art-recognized procedures from known or commercially available starting materials. If the starting materials are unavailable from a commercial source, their synthesis is described herein, or they can be prepared by procedures known in the art.

Specifically, the compounds of formula (I) may be prepared by processes comprising:

a) reductive amination of an aldehyde of formula R⁶CHO wherein R⁶ is C₀₋₂ alkylAr¹ where Art is as defined in formula (I) with 1-deoxygalactonojirimycin [2-(hydroxymethyl)-3,4,5-piperidinetriol, (2R,3S,4R,5S)] (II) which is commercially available:

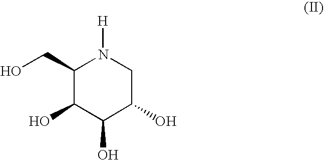

The reductive amination may be performed by methods known to those skilled in the art, e.g. using NaBH₃CN or a supported reagent such as (polystyrylmethyl) trimethylammonium cyanoborohydride in acetic acid-methanol or HCl-methanol, or using NaBH(OAc)₃ in a solvent, such as dichloromethane-methanol. Alternatively, the reductive amination may be performed using hydrogen in the presence of a catalyst, e.g. palladium on carbon, in the presence of an acid, e.g. acetic acid, in a suitable solvent such as ethanol;

b) alkylation of 1-deoxygalactonojirimycin (1) with an activated species R⁶CH₂X wherein R⁶ is as defined above and X is a leaving group, e.g. Cl, Br or I, or a sulfonyloxy group e.g. mesyloxy, tosyloxy. Such a reaction can be carried out in a solvent such as DMF in the presence of a base, e.g. pyridine; or
c) N-acylation of a suitably protected derivative of 1-deoxygalactonojirimycin (II) with an activated acyl derivative e.g. an acyl halide, acyl anhydride or mixed anhydride followed by reduction of the resultant amide with a reducing agent such as lithium aluminium hydride, in a suitable solvent e.g. THF, followed by deprotection of the resulting compound of formula (M):

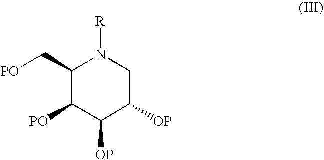

wherein R is as defined in formula (I) and P, which may be the same or different, are hydroxy protecting groups e.g. benzyl or substituted benzyl. When P is benzyl or substituted benzyl the deprotection is preferably conducted in the presence of hydrogen gas and a catalyst, such as, PdCl₂ or palladium on carbon in a suitable solvent, such as, an alcohol, e.g. ethanol. It will be understood that when P is benzyl or substituted benzyl and R is substituted benzyl, the R group can also be removed under these conditions to give compounds of formula (II). Thus, compounds of formula (I) where R is substituted benzyl are preferably produced using process a) or b) above.

The invention also provides a compound of formula (I) when produced according to the methods described above.

During the synthesis of the compounds of formula (I) labile functional groups in the intermediate compounds, e.g. hydroxy, carboxy and amino groups, may be protected. A comprehensive discussion of the ways in which various labile functional groups may be protected and methods for cleaving the resulting protected derivatives is given in for example Protective Groups in Organic Chemistry, T. W. Greene and P. G. M. Wuts (Wiley-Interscience, New York 2nd edition, 1991).

The compounds of formula (I) may be prepared singly or as compound libraries comprising at least 2, for example 5 to 500 compounds and more preferably 10 to 100 compounds of formula (I). Libraries of compounds of formula (I) may be prepared by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable salts or prodrugs thereof.

The pharmaceutically acceptable salts and prodrugs of the compounds of formula (I) may be prepared by methods well known to those skilled in the art.

Any novel intermediate compounds as described herein also fall within the scope of the present invention. Thus according to a further aspect of the invention there is provided a compound of formula (III) as defined above.

The pharmaceutically effective compounds of formula (I) may be administered in conventional dosage forms prepared by combining a compound of formula (I) (“active ingredient”) with standard pharmaceutical carriers, excipients or diluents according to conventional procedures well known in the art. These procedures may involve mixing, granulating and compressing or dissolving the ingredients as appropriate to the desired preparation.

According to a further aspect, the present invention provides a pharmaceutical composition comprising a compound of formula (I), together with one or more pharmaceutically acceptable carriers, excipients and/or diluents.

The active ingredient or pharmaceutical composition can be administered simultaneously, separately or sequentially with another treatment for the disorder to be treated.

The active ingredient or pharmaceutical composition may be administered to a subject by any of the routes conventionally used for drug administration, for example they may be adapted for oral (including buccal, sublingual), topical (including transdermal), nasal (including inhalation), rectal, vaginal or parenteral (including subcutaneous, intramuscular, intravenous or intradermal) administration to mammals including humans. The most suitable route for administration in any given case will depend on the particular compound or pharmaceutical composition, the subject and the nature and severity of the disease and the physical condition of the subject. Such compositions may be prepared by any method known in the art of pharmacy, for example by bringing into association the active ingredient with the carrier(s), excipient(s) and/or diluent(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Tablets and capsules for oral administration may be in unit dose presentation form and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Pharmaceutical compositions adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams. Such applications include those to the eye or other external tissues, for example the mouth and skin and the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. The composition may also contain compatible conventional carriers, such as cream or ointment bases and ethanol or oleyl alcohol for lotions.

Pharmaceutical compositions adapted for topical administration to the eye include eye drops wherein the active ingredient is dissolved or suspended in a suitable carrier, especially an aqueous solvent.

Pharmaceutical compositions adapted for topical administration in the mouth include lozenges, pastilles and mouthwashes.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318, (1986).

Pharmaceutical compositions adapted for nasal administration wherein the carrier is a solid include a coarse powder having a particle size for example in the range 20 to 500 microns which is administered by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurised aerosols, nebulizers or insufflators.

Pharmaceutical compositions adapted for rectal administration may be presented as suppositories or enemas. Suppositories will contain conventional suppository bases, e.g. cocoa-butter or other glyceride.

Pharmaceutical compositions adapted for vaginal administration may be presented as pessaries, tampons, creams, gels, pastes, foams or spray compositions.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient, and aqueous and non-aqueous sterile suspensions which may include suspending agents and thickening agents. The compositions may be presented in unit-dose or multi-dose containers, for example sealed ampoules and vials and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

For parenteral administration, fluid unit dosage forms are prepared utilizing the active ingredient and a sterile vehicle, e.g. water. The active ingredient, depending on the vehicle and concentration used, can be either suspended or dissolved in the vehicle. In preparing solutions the active ingredient can be dissolved in water for injection and filter sterilised before filling into a suitable vial or ampoule and sealing.

Advantageously, agents such as a local anaesthetic, preservative and buffering agents can be dissolved in the vehicle. To enhance stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the active ingredient is suspended in the vehicle instead of being dissolved and sterilization cannot be accomplished by filtration. The active ingredient can be sterilised by exposure to ethylene oxide before suspending in the sterile vehicle. Advantageously, a surfactant or wetting agent is included in the composition to facilitate uniform distribution of the active ingredient.

The pharmaceutical compositions according to the invention are preferably adapted for oral administration.

It should be understood that in addition to the ingredients particularly mentioned above, the compositions may also include other agents conventional in the art having regard to the type of formulation in question, for example those suitable for oral administration may include flavouring agents. They may also contain therapeutically active agents in addition to the compounds of the present invention. Such carriers may be present as from about 1% up to about 98% of the formulation. More usually they will form up to about 80% of the formulation.

The compositions may contain from 0.1% by weight, e.g. 10-60% by weight, of the active material, depending on the method of administration.

Pharmaceutical compositions may be presented in unit dose forms containing a predetermined amount of active ingredient per dose. Such a unit may contain for example 0.1 mg/kg to 750 mg/kg, more preferably 0.1 mg/kg to 10 mg/kg depending on the condition being treated, the route of administration and the age, weight and condition of the patient. Preferred unit dosage compositions are those containing a daily dose or sub-dose, or an appropriate fraction thereof, of an active ingredient.

It will be recognized by one of skill in the art that the optimal quantity and spacing of individual dosages of active ingredients will be determined by the nature and extent of the condition being treated, the form, route and site of administration and the particular subject being treated and that such optimums can be determined by conventional techniques. It will also be appreciated by one of skill in the art that the optimal course of treatment, i.e. the number of doses of the active ingredients given per day for a defined number of days, can be ascertained by those skilled in the art using conventional course of treatment determination tests.

No toxicological effects are indicated when the compounds of formula (I) are administered in the above mentioned dosage range.

The compounds of the invention are useful in that they are capable of inhibiting glucosylceramide synthase. Thus, the compounds of the invention can be used in the treatment of various glycolipid storage diseases, such as, Gaucher's disease, Sandhoffs disease, Tay-Sachs disease, Fabry disease, GM1 gangliosidosis etc. In addition, compounds, such as this also can find use in the treatment of conditions in which glycolipid accumulation occurs, such as Niemann-Pick disease, mucopolysaccharidoses (MPS I, MPS MA, MPS IIIB, MPS VI and MPS VII, preferably MPS I), mucolipidosis type IV and α-mannosidosis.

The compounds of the present invention can also be used in the treatment of cancers in which glycolipid synthesis is abnormal, such as brain tumours, neuroblastoma, malignant melanoma, renal adenocarcinoma and multi-drug resistant cancers in general.

The compounds of the present invention can also be used in the treatment of diseases caused by infectious organisms which use cell surface glycolipids as receptors for either the infectious organism itself or for a toxin produced by the infectious organism (e.g. for attachment and/or invasion onto/into the host cell).

The compounds of the present invention may also be used in the treatment of diseases caused by infectious organisms for which the synthesis of glucosylceramide is an essential or important process, such as pathogenic fingi, e.g. Cryptococcus neoformans or viral infections, e.g. viruses that require host cell enzymes to synthesize and properly fold their viral envelope glycoproteins, or viruses that acquire a component of their envelope from an internal host cell membrane. GCS inhibition may result in improper glycoprocessing or the misfolding of one or more viral envelope glycoproteins, inhibition of viral secretion, or improper viral fusion of the virus with its target cells. Suitable viral infections for treatment may be caused by, for example but not limited to, the following viruses: flaviviruses and pestiviruses, e.g. hepatitis C virus, yellow fever virus, dengue viruses 14, Japanese encephalitis virus, Murray Valley encephalitis virus, Rocio virus, West Nile fever virus, St. Louis encephalitis virus, tick-borne encephalitis virus, Louping ill virus, Powassan virus, Omsk hemorrhagic fever virus, and Kyasanur forest disease virus; hepadnavirus, e.g. hepatitis B virus; paramyxovirus, e.g. respiratory syncytial virus or retroviruses, such as human immunodeficiency virus.

The compounds of the present invention can also be used in the treatment of diseases in which excessive glycolipid synthesis occurs, such as, but not limited to, atherosclerosis, polycystic kidney disease and diabetic renial hypertrophy.

The compounds of the present invention can also be used in the treatment of neuronal disorders, such as Alzheimer's disease or epilepsy; and neuronal degenerative diseases, such as Parkinsons' disease.

The compounds of the present invention can also be used in the treatment of neuronal injury, such as spinal cord injuries or stroke.

The compounds of the present invention can also be used for reversibly rendering a male mammal infertile.

The compounds of the present invention can also be used in the treatment of obesity.

The compounds of the present invention can also be used in the treatment of inflammatory diseases or disorders associated with macrophage recruitment and activation, including but not limited to, rheumatoid arthritis, Crohn's disease, asthma and sepsis.

In additional aspects, therefore, the present invention provides:

(i) the use of a compound of formula (I) in the manufacture of a medicament for use as an inhibitor of glucosylceramide synthase.

(ii) the use of a compound of formula (I) in the manufacture of a medicament for the treatment of a glycolipid storage disease. Examples of glycolipid storage disease which can be treated include, but are not limited to, Gaucher disease, Sandhoffs disease, Tay-Sachs disease, Fabry disease or GM1 gangliosidosis.

(iii) the use of a compound of formula (I) in the manufacture of a medicament for the treatment of Niemann-Pick disease, types A and C.

(iv) the use of a compound of formula (I) in the manufacture of a medicament for the treatment of mucopolysaccharidosis type I, mucopolysaccharidosis type IIIA, mucopolysaccharidosis type IIIB, mucopolysaccharidosis type VI or mucopolysaccharidosis type VII. Preferably the compounds are used in the treatment of mucopolysaccharidosis type I.

(v) the use of a compound of formula (I) in the manufacture of a medicament for the treatment of α-mannosidosis or mucolipidosis type V.

(vi) the use of a compound of formula (I) in the manufacture of a medicament for the treatment of cancer in which glycolipid synthesis is abnormal, including but not limited to brain cancer, neuronal cancer, neuroblastoma, renal adenocarcinoma, malignant melanoma, multiple myeloma and multi-drug resistant cancers.

(vii) the use of a compound of formula (I) in the manufacture of a medicament for use in the treatment of Alzheimer's disease, epilepsy or stroke.

(viii) the use of a compound of formula (I) in the manufacture of a medicament for use in the treatment of Parkinson's disease.

(ix) the use of the compound of formula (I) in the manufacture of a medicament in the treatment of spinal injury.

(x) the use of a compound of formula (I) in the manufacture of a medicament for use in the treatment of diseases caused by infectious microorganisms which utilize glycolipids on the surface of cells as receptors for either the organism itself or for toxins produced by the organism.

(xi) the use of a compound of formula (I) in the manufacture of a medicament for use in the treatment of disease caused by infectious organisms for which the synthesis of glucosylcermide is an essential or important process, such as but not limited to, pathologies associated with infections of pathogenic fungi, e.g. Cryptococcus neoformans or pathologies associated with viral infections.

(xii) the use of a compound of formula (I) in the manufacture of a medicament for use in the treatment of diseases associated with abnormal glycolipid synthesis, including but not limited to, polycystic kidney disease, diabetic renal hypertrophy and atherosclerosis.

(xiii) the use of a compound of formula (I) in the manufacture of a medicament for the treatment of a condition treatable by the administration of a ganglioside, such as GM1 ganglioside. Examples of such conditions are Parkinson's disease, stroke and spinal cord injuries.

(xiv) the use of a compound of formula (I) in the manufacture of a medicament for reversibly rendering a male mammal infertile.

(xv) the use of a compound of formula (I) in the manufacture of a medicament for the treatment of obesity, e.g. as an appetite suppressant.

(xvi) the use of a compound of formula (I) in the manufacture of a medicament for the treatment of inflammatory diseases or disorders associated with macrophage recruitment and activation, including but not limited to, rheumatoid arthritis, Crohn's disease, asthma and sepsis.

(xvii) a method for the treatment of a glycolipid storage disease, e.g. Gaucher's disease, Sandhoffs disease, Tay-Sachs disease or GM1 gangliosidosis, which comprises the step of administering to a patient an effective amount of a compound of formula (I).

(xviii) a method for the treatment of Niemann-Pick disease, types A and C, which comprises the step of administering to a patient an effective amount of a compound of formula (I).

(xix) a method for the treatment of mucopolysaccharidosis type I, mucopolysaccharidosis type MA, mucopolysaccharidosis type DIB, mucopolysaccharidosis type VI or mucopolysaccharidosis type VII which comprises the step of administering to a patient an effective amount of a compound of formula (I).

(xx) a method for the treatment of α-mannosidosis or mucolipidosis type IV which comprises the step of administering to a patient an effective amount of a compound of formula (I).

(xxi) a method for the treatment of cancer in which glycolipid synthesis is abnormal, including but not limited to brain cancer, neuronal cancer, renal adenocarcinoma, malignant melanoma, multiple myeloma and multi-drug resistant cancers, which comprises the step of administering to a patient an effective amount of a compound of formula (I).

(xxii) a method for the treatment of Alzheimer's disease, epilepsy or stroke which comprises the step of administering to a patient an effective amount of a compound of formula (I).

(xxiii) a method for the treatment of Parkinson's disease, which comprises the step of administering to a patient an effective amount of a compound of formula (I).

(xxiv) a method for the treatment of spinal injury which comprises the step of administering to a patient an effective amount of a compound of formula (I).

(xxv) a method for the treatment of diseases caused by infectious microorganisms, which utilize glycolipids on the surface of cells as receptors for either the organism itself or for toxins produced by the organism, which comprises the step of administering to a patient an effective amount of a compound of formula (I).

(xxvi) a method for the treatment of diseases caused by infectious organisms, e.g. pathogenic fungi or viruses, for which the synthesis of glucosylceamide is an essential or important process, such as but not limited to, pathologies associated with Cryptococcus neoformans infection, or pathologies associated with viral infections, which comprises the step of administering to a patient an effective amount of a compound of formula (I).

(xxvii) a method for the treatment of diseases associated with abnormal glycolipid synthesis including but not limited to polycystic kidney disease, diabetic renal hypertrophy and atherosclerosis, which comprises the step of administering to a patient an effective amount of a compound of formula (I).

(xxviii) a method for the treatment of a condition treatable by the administration of a ganglioside, such as GM1 ganglioside, which comprises the step of administering to a patient an effective amount of a compound of formula (I). Examples of such conditions are Parkinson's disease, stroke and spinal cord injuries.

(xxix) a method for reversibly rendering a male mammal infertile, which comprises the step of administering to said male mammal an effective amount of a compound of formula (I).

(xxx) a method for the treatment of obesity, which comprises the step of administering to a patient an effective amount of a compound of formula (I).

(xxxi) a method for the treatment of inflammatory diseases or disorders associated with macrophage recruitment and activation, including but not limited to, rheumatoid arthritis, Crohn's disease, asthma and sepsis, which comprises the step of administering to a patient an effective amount of a compound of formula (I).

The invention also provides for the use of a compound of formula (I) for the treatment of the above-mentioned diseases and conditions.

All publications, including, but not limited to, patents and patent applications, cited in this specification, are herein incorporated by reference as if each individual publication were specifically and individually indicated to be incorporated by reference herein as though fully set forth.

The invention will now be described by reference to the following Examples, which are merely illustrative and are not to be construed as a limitation of the scope of the present invention.

### EXAMPLE 1

### 3,4,5-Piperidinetriol, 2(hydroxymethyl)-1-[[4-phenylmethoxy)phenyl]methyl]-, (2R,3S,4R,5S)

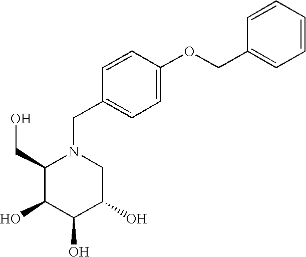

To a stirred solution of 3,4,5-piperidinetriol, 2-(hydroxymethyl)-, (2R,3S,4R,5S) (52 mg, 0.32 mmol), acetic acid (34 mg, 0.57 mmol) and 4-(phenylmethoxy)benzaldehyde (210 mg, 1.06 mmol) in methanol (3 ml), was added (polystyrylmethyl)-trimethylammonium cyanoborohydride (180 mg, 0.77 mmol). After stirring for 24 h, further acetic acid (54 mg, 0.90 mmol) was added and the reaction mixture was stirred for a further 5 days. The resin was filtered off and the solution was diluted with ˜4 times its volume of water and then made basic by the addition of concentrated aqueous ammonia. The resulting emulsion was loaded onto a short plug of hydrophobic resin (Supelco Diaion HP-20SS, 1 g). [The resin had been wetted with methanol and then equilibrated with water prior to its use]. The resin was eluted with water and then the product was washed off using 50-80% methanol in water. The product containing solution was loaded directly onto a plug of acidic Dowex 50×4-200 resin (1.5 g). The resin was eluted with methanol (20 ml) to remove non-basic side products. The title compound was then eluted using a mixture of methanol (20 ml) and concentrated ammonium hydroxide (10 ml). The resulting solution was concentrated to a small volume (1 ml) and then freeze dried to afford the title compound as a white solid (50 mg, 43%). ¹H NMR (d4-methanol) δ 1.81 (1H, t, J=10.7 Hz), 2.35-2.41 (1H, m), 2.88 (1H, dd, J=4.9, 11.3 Hz), 3.18-3.36 (2H, m), 3.73 (1H, ddd, J=4.9, 9.8, 9.8 Hz), 3.88-4.06 (4H, m), 5.07 (2H, s), 6.94 (2H, d, J=8.7 Hz), 7.24-7.46 (7H, m). MS m/z/z 360.2 (M+H)⁺.

### Biological Assays

The compounds of the invention may be tested for their biological activity in the following assays:

Inhibition of GCS

The assay for inhibition of GCS was performed essentially as described in Platt et al., J. Biol. Chem., (1994), 269, 27108, the enzyme source being human recombinant GCS expressed in insect cells.

Inhibition of Non-lysosomal-3-β-glucocerebrosidase

The assay for inhibition of non-lysosomal-β-glucocerebrosidase was essentially carried out as described in Overkleeft, H. S. et al., J. Biol. Chem., (1998) 273, 26522-26527 with the following differences: whole cell extracts of MCF7 (a human breast carcinoma cell line) was used as the source of the enzyme instead of splenic membrane suspensions; 5 mM instead of 3 mM, 4-MU β-glucoside was used as substrate and 0.2M citrate/phosphate (pH 5.8) was used instead of McIlvaine buffer.

Table I shows IC₅₀ data for compounds of the invention against human GCS and non-lysosomal-β-glucocerebrosidase enzymes.

TABLE I

| | | Inhibition of non-lysosomal-β- |
|---|---|---|
| | Inhibition of GCS | glucocerebrosidase |
| Compound | (IC₅₀ μM) | (IC₅₀ μM) |
| Example 1 | 1.3 | 10 |

Estimating the Cell-Based IC₅₀ for GCS Inhibition by Measuring Glucosylceramide (GlcCer) Depletion

Human mammary epithelial cells (MCF-7) are cultured for 5-7 days, with varying concentrations of a compound of the invention to be tested (0; 0.01; 0.05; 0.25; 1.25 and 6.25 μM). The cells are harvested and the total cellular lipids extracted. Neutral glycolipids are separated by partitioning in a DIPE/1-butanol/saline suspension, according to methods well known to those skilled in the art. The neutral glycolipid extracts are then separated by High-Performance Thin Layer Chromatography (HPJC), using non-polar TLC conditions (chloroform:methanol: 0.2% CaCl₂; 65:35:8), according to methods well known to those skilled in the art. GlcCer bands are visualized and the TLC plates were scanned immediately. Scion Image software is then used to quantify GlcCer in the samples relative to a GlcCer standard. This enables a cell-based IC₅₀ to be calculated for compounds of the invention for GCS inhibition.

1. A compound of formula (I) or a pharmaceutically acceptable salt or prodrug thereof: 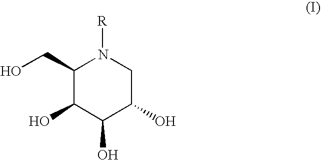
wherein
R is C₁₋₃ alkylAr¹ where Ar¹ is phenyl or pyridyl;
wherein phenyl is substituted by one or more substituents selected from CN, CON(R¹)₂, SOₙR²SO₂N(R¹)₂, N(R⁵)₂, N(R¹)COR², N(R¹)SOₙR², C₀₋₆alkylAr², C₂₋₆ alkenylAr² and C₃₋₆ alkynylAr² wherein one or more of the —CH₂— groups of the alkyl chain may be replaced with a heteroatom selected from O, S and NR³, provided that when the heteroatom is O, at least two —CH₂— groups separate it from any additional O atom in the alkyl chain; or two adjacent substituents on the Ar¹ phenyl may together form a fused 5- or 6-membered saturated or unsaturated ring wherein the ring optionally contains 1 or 2 heteroatoms selected from O, S and NR⁴ and is optionally substituted by one or more substituents selected from, an oxo group, C₁₋₆ alkyl and C₀₋₃ alkylAr⁴;
and the Ar¹ phenyl is optionally substituted by one or more additional substituents selected from F, Cl, Br, CF₃, OCF₃, OR³ and C₁₋₆alkyl;
and wherein pyridyl is substituted by one or more substituents, selected from, CN, CON(R¹)₂, SOₙR²SO₂N(R¹)₂, N(R⁵)₂, N(R¹)COR², N(R¹)SOₙR², F, Cl, Br, CF₃, OCF₃, OR³, C₁₋₆ alkyl, C₀₋₆ alkylAr², C₂₋₆ alkenylAr² and C₃₋₆ alkynylAr² wherein one of the —CH₂— groups of the alkyl chain may be replaced with a heteroatom selected from O, S and NR³, provided that when the heteroatom is 0, at least two —CH₂— groups separate it from any additional O atom in the alkyl chain; or two adjacent substituents on the Ar¹ pyridyl may together form a fused 5- or 6-membered saturated or unsaturated ring wherein the ring optionally contains 1 or 2 heteroatoms selected from O, S and NR⁴ and is optionally substituted by one or more substituents selected from, an oxo group, C₁₋₆ alkyl and C₀₋₃ alkylAr⁴;
R¹ is H, C₁₋₆ alkyl optionally substituted by OH, Ar³, or C₁₋₆ alkylAr³, or the group N(R¹)₂ may form a 5- to 10-membered heterocyclic group optionally containing one or more additional heteroatoms selected from O, S and NR³ and is optionally substituted by an oxo group;
R² is C₁₋₆ alkyl optionally substituted by OH, Ar³, or C₁₋₆ alkylAr³;
R³ is H, or C₁₋₆ alkyl;
R⁴ is H, C₁₋₆ alkyl or C₀₋₃ alkylAr⁴;
R⁵ is H, C₁₋₆ alkyl optionally substituted by OH, Ar³, or C₁₋₆ alkylAr³, or the group N(R⁵)₂ may form a 5- to 10-membered heterocylic group optionally containing one or more additional heteroatoms selected from O, S and NR³ and is optionally substituted by an oxo group;
Ar² and Ar³ are independently phenyl or a 5- to 10-membered heteroaryl group containing up to 3 heteroatoms selected from O, S and NR³, which may be optionally substituted by one or more substituents selected from F, Cl, Br, CN, CF₃, OCF₃, OR³ and C₁₋₆ alkyl;
Ar⁴ is phenyl or pyridyl either of which may be optionally substituted by one or more substituents selected from F, Cl, Br, CN, CF₃, OCF₃, OR³ and C₁₋₆ alkyl; and
n=0, 1 or 2;
provided that the compound is not:
a) 3,4,5-piperidinetriol, 1-[(1,1′-biphenyl)-4-ylmethyl]-2-(hydroxymethyl)-, (2R,3S,4R,5S);
b) 3,4,5-piperidinetriol, 2-(hydroxymethyl)-1-[(4-methoxyphenyl)methyl]-, (2R,3S,4R,5S);
c) 3,4,5-piperidinetriol, 2-(hydroxymethyl)-1-[(4-methylthiophenyl)methyl]-, (2R,3S,4R,5S);
d) acetamide, N-[4-[[3,4,5-trihydroxy-2-(hydroxymethyl)-1-piperidinyl]methyl]phenyl]-, (2R,3S,4R,5S); or
e) 3,4,5-piperidinetriol, 2-(hydroxymethyl)-1-[(4-methoxy-3-methylphenyl)methyl]-, (2R,3S,4R,5S).

2. A compound as defined in claim 1 wherein R is C₁ alkylAr¹.

3. A compound as defined in claim 1 wherein Ar¹ is phenyl.

4. A compound as defined in claim 1, wherein Ar¹ is phenyl substituted by one or more substituents selected from CN, CON(R¹)₂, N(R⁵)₂, and C₀₋₆ alkylAr², wherein one or more of the —CH₂— groups of the alkyl chain may be replaced with a heteroatom selected from O, S and NR³, provided that when the heteroatom is 0, at least two —CH₂— groups separate it from any additional O atom in the alkyl chain, or two adjacent substituents on the Ar¹ pyridyl may together form a fused 5- or 6-membered saturated or unsaturated ring wherein the ring optionally contains 1 or 2 heteroatoms selected from O and NR⁴ and is optionally substituted by one or more substituents selected from, an oxo group, C₁₋₆ alkyl and C₀₋₃ alkylAr⁴; and the Ar¹ phenyl is optionally substituted by one or more additional substituents selected from F, Cl, Br, CF₃, OCF₃, OR³ and C₁₋₆ alkyl.

5. A compound as defined in claim 1, wherein Ar¹ is phenyl substituted by one or more substituents selected from CN, CON(R¹)₂, N(R⁵)₂, and C₀₋₆ alkylAr² wherein one or more of the —CH₂— groups of the alkyl chain may be replaced with O, provided that at least two —CH₂— groups separate it from any additional O atom introduced into the alkyl chains and the Ar¹ phenyl is optionally substituted by one or more additional substituents selected from F, Cl, Br, CF₃, OCF₃, OR³ and C₁₋₆ alkyl.

6. A compound as defined in claim 1, wherein Ar² is phenyl which is optionally substituted by one or more substituents selected from F, Cl, Br, CN, CF₃, OCF₃, OR³ and C₁₋₆ alkyl.

7. A compound as defined in claim 1, wherein R¹ is H or C₁₋₆ alkylAr³.

8. A compound as defined in claim 1, wherein R⁴ is H or C₁₋₆ alkyl.

9. A compound as defined in claim 1, wherein Ar³ is phenyl which may be optionally substituted by one or more substituents selected from F, Cl, Br, CN, CF₃, OCF₃, OR³ and C₁₋₆ alkyl.

10. A compound as defined in claim 1, wherein R⁵ is C₁₋₆ alkyl.

11. A compound of formula (I) as described in Example 1 or a pharmaceutically acceptable salt or prodrug thereof.

12. (canceled)

13. A pharmaceutical composition comprising a compound of formula (I) as defined in claim 1, together with one or more pharmaceutically acceptable carriers, excipients and/or diluents.

14. A process for the preparation of a compound of formula (I) as defined in claim 1, which process comprises:
a) conducting reductive amination of an aldehyde of formula R⁶CHO, wherein R⁶ is C₀₋₂ alkylAr¹ where Ar¹ is as defined in claim 1 with 1-deoxygalactonojirimycin [2-(hydroxymethyl)-3,4,5-piperidinetriol, (2R,3S,4R,5S)] (II): 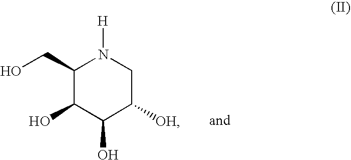
b) alkylating 1-deoxygalactonojirimycin (II) with an activated species R⁶CH₂X, wherein R⁶ is as defined above and X is a leaving group; or
c) N-acylating a protected derivative of 1-deoxygalactonojirimycin (II) with an activated acyl derivative, followed by reduction of the resultant amide with a reducing agent and deprotection.

15-29. (canceled)

30. A compound of formula (III): 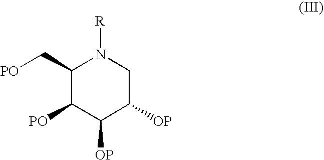
wherein R is as defined in claim 1 and P, which may be the same or different, are hydroxy protecting groups.

31. A method for preventing or treating a glycolipid storage disease, comprising administering to a subject in need thereof the compound of claim 1.

32. The method of claim 31, wherein said glycolipid storage disease is Gaucher disease, Sandhoffs disease, Tay-Sachs disease, Fabry disease or GM1 gangliosidosis.

33. A method for preventing or treating a disorder or disease associated with abnormal glycolipid synthesis, comprising administering to a subject in need thereof the compound of claim 1.

34. The method of claim 33, wherein said disorder or disease is cancer.

35. The method of 34, wherein said cancer is selected from the group consisting of brain cancer, neuronal cancer, neuroblastoma, renal adenocarcinoma, malignant melanoma, multiple myeloma and multi-drug resistant cancer.

36. A method for preventing or treating a disorder or disease associated with abnormal glycolipid metabolism, comprising administering to a subject in need thereof the compound of claim 1.

37. The method of claim 36, wherein said disorder or disease is Niemann-Pick disease type C, mucopolysaccharidosis type I, mucopolysaccharidosis type IIIA, mucopolysaccharidosis type IIIB, mucopolysaccharidosis type VI, mucopolysaccharidosis type VII, α-mannosidosis or mucolipidosis type IV.

38. The method of claim 36, wherein said disorder or disease is selected from the group consisting of Alzheimer's disease, epiliepsy, stroke, Parkinson's disease and spinal injury.

39. A method for preventing or treating an infectious disease caused by an infectious microorganism that utilizes glycolipids on the surface of cells as receptors for either said microorganism itself or for toxins produced by said microorganism, or by an infectious microorganism that requires the synthesis of glucosylceramide to sustain an infection, said method comprising administering to a subject in need thereof the compound of claim 1.

40. A method for preventing or treating a condition treatable by the administration of a ganglioside, said method comprising co-administering to a subject in need thereof said ganglioside and the compound of claim 1.

41. The method of claim 40, wherein said ganglioside is a GM1 ganglioside.

42. A method for reversibly rendering a male mammal infertile, comprising administering to said mammal the compound of claim 1.

43. A method for treating obesity, comprising administering to a subject in need thereof the compound of claim 1.

44. A method for treating an inflammatory disorder or disease associated with macrophage recruitment and activation, comprising administering to a subject in need thereof the compound of claim 1.

45. The method of claim 44, wherein said inflammatory disorder or disease is selected from the group consisting of rheumatoid arthritis, Crohn's disease, asthma and sepsis.
